(19) United States
(12) Patent Application Publication
(10) Pub. No.: US 2023/0240893 A1
(43) Pub. Date: Aug. 3, 2023

(54) LASER FEEDBACK DEVICE IRRADIATED TO EYEBALL AND LASER FEEDBACK METHOD USING SAME

(71) Applicant: THE CATHOLIC UNIVERSITY OF KOREA INDUSTRY-ACADEMIC COOPERATION FOUNDATION, Seoul (KR)

(72) Inventors: Dong Jin CHANG, Seoul (KR); Young Jung ROH, Seoul (KR)

(21) Appl. No.: 18/012,606

(22) PCT Filed: Jan. 21, 2021
(86) PCT No.: PCT/KR2021/000807

§ 371 Date: Dec. 22, 2022

(30) Foreign Application Priority Data
Jun. 23, 2020 (KR) 10-2020-0076250

(51) Int. Cl. A61F 9/008; A61B 3/12

(52) U.S. Cl. CPC A61F 9/008; A61B 3/1225; A61B 2018/2253; A61F 2009/00844; A61F 2009/00863; A61F 2009/00885

(57) ABSTRACT

Proposed are a laser feedback device for irradiation to an eyeball and a laser feedback method using the same, the laser feedback device comprising: a guide beam irradiation unit for irradiating a guide beam to an eyeball of a patient; a reflection unit positioned adjacent to the guide beam irradiation unit; a laser irradiation unit for irradiating a laser into the eyeball; an image acquisition unit that receives a reflection beam reflected from the eyeball and refracted through the reflection unit and acquires at least one retinal image of the retina of the eyeball; and an image mapping unit that maps at least one retinal image to construct a macular image which is a full image of the macula of the eyeball.

### TECHNICAL FIELD

The present invention relates to a laser feedback device for irradiating an eyeball and a laser feedback method using the same, and more particularly, to a laser feedback device for irradiating an eyeball, which can identify a lesion position and an irradiation position of laser irradiated to the eyeball by mapping at least one retinal image and a laser feedback method using the same.

### BACKGROUND ART

Diseases related to tissues composed of layered structure occur in a wide variety of forms. In particular, the main part of a retinal tissue of an eyeball is composed of a layered structure.

When the intraocular tissue having such a layered structure is structurally deformed, it is very common that visual acuity is deteriorated or, seriously, vision loss occurs.

Cases of vision loss due to macular degeneration and related macular diseases have surpassed the rate of vision loss due to conventional glaucoma or cataracts to become the number one cause of vision loss. In particular, according to the National Health Insurance Corporation, the number of patients with macular degeneration among young people in their 20s and 40s nearly doubled from 7,631 in 2000 to 13,673 in 2004.

Macular degeneration has no symptoms, but the vision gradually deteriorates, eventually leading to vision loss. In the early stages, objects appear blurry or near objects appear distorted, but symptoms such as vision loss appear only after the disease has progressed considerably.

These macular-related diseases are the main causes of vision loss, such as retinopathy of prematurity in children, diabetic retinopathy in adults, and age-related macular degeneration. There are slight differences when considering their underlying diseases as the cause of visual loss, but in common, these diseases are known to show edema in the central part of the retina due to secondary retinal vascular endothelial cell proliferation due to damage to a blood-retinal barrier, bleeding caused by the growth of useless blood vessels in the macula, resulting in severe visual impairment.

In particular, among the various types of diseases that are emerging with the explosive increase in the aging population worldwide, the population with reduced vision and vision loss due to macular degeneration is estimated to account for more than 10% of those over 65 years of age. It is predicted that more and more people will suffer from visual impairment due to macular degeneration in the future.

Currently, there are two main types of treatment for macular diseases: surgery and drug therapy. On the other hand, it is known that there is no cure for vision loss occurring in the macula, although the key to treatment is to suppress the formation of new blood vessels in the choroid covering the outer retina.

For drug therapy, “Visudyne,” a photodynamic therapy marketed by Novartis, a multinational pharmaceutical company, and several types of angiogenesis inhibitors (Anti-VECF) are effective in some cases of diseases caused by macular degeneration. However, there is no known drug treatment for patients with dry macular degeneration, which is the most common case, and it has been reported that the side effects are very serious when drug therapy is used.

Specifically, there are laser photocoagulation and resection, and these methods creates laser burn on a part of the retina using Ar ion, Kr ion lasers, that is, a scar by inflicting a kind of burn, preventing any more new blood vessels from growing. However, these methods have a problem in that the size of the coagulated part is so large that it cannot be ignored compared to the size of the macula, and surgery around the macula directly related to vision cannot be performed.

In addition, in the laser therapy according to the prior art, a contrast agent is administered to determine a lesion location after the surgery and continuous fluorescein angiography is performed. However, with this method, a lesion position could not be identified after 2 weeks after the surgery, so it was difficult to re-operate if the disease in the affected area recurred.
(Patent Document 1) KR Registered Patent Publication No. 10-1243998 (2013.03.08.)
(Patent Document 2) KR Registered Patent Publication No. 10-1315736 (2013.10.01.)

### DISCLOSURE

### Technical Problem

An object of the present invention to solve the above problems is to provide a laser feedback device for irradiating an eyeball which supports to easily identify a lesion position and an irradiation position of laser irradiated to the eyeball by acquiring at least one retinal image through a laser irradiated from a laser irradiation unit, and then mapping the retinal image to construct and record a macular image, and a laser feedback method using the same.

An object of the present invention to solve the above problems is to provide a laser feedback device for irradiating an eyeball, which treats only a lesion locally by irradiating a laser having a low energy level from a laser irradiation unit so as not to form a laser burn on the lesion around the retina of a patient, and a laser feedback method using the same.

The technical objects to be achieved by the present invention are not limited to the technical objects mentioned above, and for those of ordinary skill in the art to which the present invention pertains, it will be apparent that other technical objects not mentioned can be clearly understood from the following description.

### Technical Solution

In order to achieve the above object, the configuration of the present invention provides a laser feedback device for irradiating an eyeball, including a guide beam irradiation unit that irradiates a guide beam toward an eyeball of a patient; a reflection unit that is positioned adjacent to the guide beam irradiation unit; a laser irradiation unit that irradiates a laser toward the eyeball; an image acquisition unit that receives a reflection beam reflected from the eyeball and refracted through the reflection unit and acquires at least one retinal image of a retina of the eyeball; and an image mapping unit that maps the at least one retinal image to construct a macular image which is a full image of a macula of the eyeball.

In an embodiment of the present invention, the laser irradiation unit may irradiate the laser by changing an irradiation angle and an irradiation position, and the at least one retinal image may be a retinal image at a different angle and position.

In an embodiment of the present invention, the image mapping unit may construct the macula image by mapping the at least one retinal image based on at least one of an optic nerve, blood vessel, and discolored region of the eyeball captured as the at least one retinal image.

In an embodiment of the present invention, the at least one retinal image and the macula image may include the irradiation position of the laser, and the image mapping unit may record the irradiation position of the laser.

In an embodiment of the present invention, the laser may irradiate only a pigment epithelium including a pigment cell and choroid of the retina.

In an embodiment of the present invention, when the laser is irradiated while the guide beam is irradiated to the eyeball, the image acquisition unit may acquire the at least one retinal image.

In an embodiment of the present invention, the laser irradiation unit may include a laser irradiation member that is positioned parallel to the reflection unit and irradiates the laser toward the eyeball; a laser control member that controls an operation of the laser; and a connection member that electrically connects the laser irradiation member and the laser control member, wherein the laser control member may control the operation of the laser irradiated from the laser irradiation member as an operation signal is transmitted to the laser irradiation member through the connection member.

In an embodiment of the present invention, the guide beam irradiation unit may include a guide beam irradiation member that irradiates the guide beam toward the eyeball; and a guide beam body that is coupled to the guide beam irradiation member, generates the guide beam and delivers the guide beam to the guide beam irradiation member, wherein the guide beam irradiation member, the reflection unit, and the laser beam irradiation member may be positioned to be parallel to each other.

In an embodiment of the present invention, the image acquisition unit may include a convex lens that is positioned under the reflection unit to receive and focus the reflection beam refracted by the reflection unit; a filter member that is positioned under the convex lens to filter the reflection beam focused by the convex lens; a concave lens that is positioned under the filter member to disperse the reflection beam filtered by the filter member; and an image processing member that is positioned under the concave lens to acquire the at least one retinal image based on the reflection beam dispersed from the concave lens, wherein the convex lens, the filter member, the concave lens, and the image processing member may be disposed to be perpendicular to the guide beam irradiation unit, the reflection unit, and the laser irradiation unit.

On the other hand, in order to achieve the above object, the configuration of the present invention provides a laser feedback method which uses a laser feedback device for irradiating an eyeball. The laser feedback method includes the steps of (a) positioning an eyeball of a patient so that the eyeball is positioned on a same straight line as a guide beam irradiation unit; (b) irradiating a guide beam toward the eyeball by the guide beam irradiation unit; (c) irradiating a laser toward the eyeball by a laser beam irradiation unit; (d) refracting a reflection beam reflected from the eyeball and reflecting the reflection beam to an image acquisition unit by a reflection unit; (e) receiving the reflection beam reflected from the eyeball and refracted through the reflection unit and acquiring at least one retinal image for a retina of the eyeball by an image acquisition unit; and (f) mapping the at least one retinal image to construct a macula image, which is an entire image of a macula of the eyeball by an image mapping unit.

In an embodiment of the present invention, the step (e) may include the steps of (e1) focusing the reflection beam by a convex lens of the image acquisition unit; (e2) filtering the reflection beam focused by a filter member of the image acquisition unit; (e3) dispersing the reflection beam filtered by a concave lens of the image acquisition unit; and (e4) acquiring the at least one retinal image for the retina of the eyeball based on the reflection beam dispersed by an image processing member of the image acquisition unit, wherein the at least one retinal image may include an irradiation position of the laser.

In an embodiment of the present invention, the step (f) may include the steps of (f1) receiving the at least one retinal image by the image mapping unit; (f2) mapping the at least one retinal image based on at least one of an optic nerve, blood vessel, and discolored region of the eyeball captured as the at least one retinal image by the image mapping unit; and constructing the macula image based on the at least one retinal image mapped by the image mapping unit, wherein the macula image may include an irradiation position of the laser, and the image mapping unit may record the irradiation position of the laser.

In an embodiment of the present invention, the step of acquiring, by the image acquisition unit 140, an image obtained by capturing a position of the guide beam may be further included before the step (c).

### Advantageous Effect

The effect of the present invention according to the above configuration is that the lesion position and the irradiation position of the laser irradiated to the eyeball can be easily determined by acquiring at least one retinal image through the laser irradiated from the laser irradiation unit, mapping to construct and record the macula image.

In addition, the effect of the present invention according to the configuration as described above is that only the lesion can be locally treated by irradiating a laser having a low energy level from the laser irradiation unit so as not to form a laser burn on the lesion around the retina of the patient.

It should be understood that the effects of the present invention are not limited to the above-described effects, and include all effects that can be inferred from the configuration of the invention described in the detailed description or claims of the present invention.

### DESCRIPTION OF DRAWINGS

FIG. 1 is a schematic view showing a laser feedback device for irradiating an eyeball according to an embodiment of the present invention.

FIG. 2 is a side view in one direction showing a guide beam irradiation unit of a laser feedback device for irradiating an eyeball according to an embodiment of the present invention.

FIG. 3 is a structural view showing 10 layers of a retina for describing a laser feedback device for irradiating an eyeball according to an embodiment of the present invention.

FIG. 4 is a view showing an eyeball and a macula for describing a laser feedback device for irradiating an eyeball according to an embodiment of the present invention.

### BEST MODE FOR INVENTION

The most preferred embodiment according to the present invention includes a guide beam irradiation unit that irradiates a guide beam to an eyeball of a patient, a reflection unit that is positioned adjacent to the guide beam irradiation unit a laser irradiation unit that irradiates a laser to the eyeball, an image acquisition unit that receives a reflection beam reflected from the eyeball and refracted through the reflection unit and acquires at least one retinal image of a retina of the eyeball, and an image mapping unit that maps the at least one retinal image to construct a macular image which is a full image of a macula of the eyeball.

### MODE FOR INVENTION

Hereinafter, the present invention will be described with reference to the accompanying drawings. However, the present invention may be embodied in several different forms, and thus is not limited to the embodiments described herein. Further, in order to clearly describe the present invention in the drawings, parts irrelevant to the description are omitted, and similar reference numerals are attached to similar parts throughout the specification.

Throughout the specification, when a part is “connected (accessed, contacted, coupled)” with another part, it includes not only “directly connected” but also “indirectly connected” with another member interposed therebetween. In addition, when a part “includes” a certain component, this means that other components may be further included, rather than excluding other components, unless otherwise stated.

The terminology used herein is used only to describe specific embodiments, and is not intended to limit the present invention. The singular expression includes the plural expression unless the context clearly dictates otherwise. In the present specification, terms such as “comprise” or “have” are intended to designate that a feature, number, step, operation, component, part, or combination thereof described in the specification exists, and it should be understood that it does not preclude the possibility of addition or existence of one or more other features, numbers, steps, operations, components, parts, or combinations thereof.

Hereinafter, embodiments of the present invention will be described in detail with reference to the accompanying drawings.

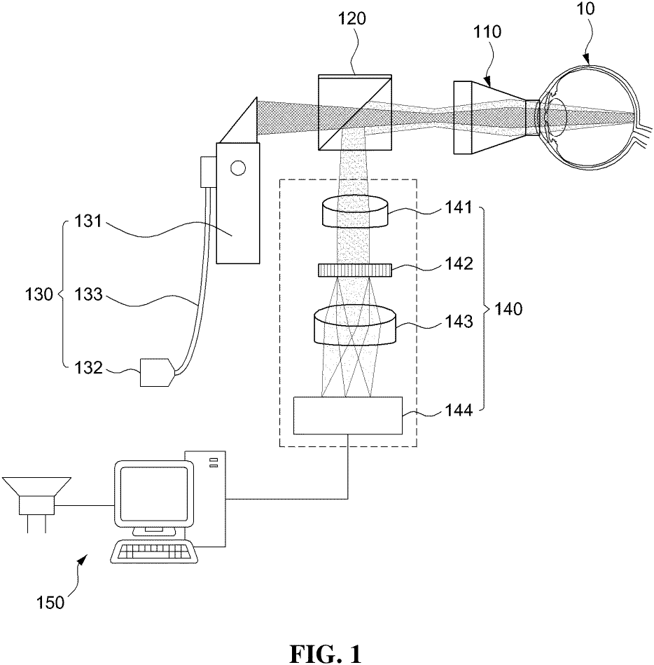

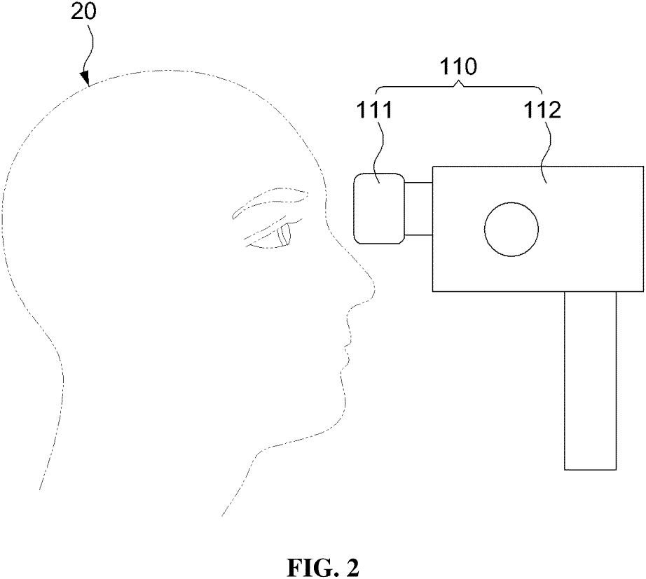

FIG. 1 is a schematic view showing a laser feedback device for irradiating an eyeball according to an embodiment of the present invention. FIG. 2 is a side view in one direction showing a guide beam irradiation unit of a laser feedback device for irradiating an eyeball according to an embodiment of the present invention.

A laser feedback device 100 for irradiating an eyeball according to an embodiment of the present invention includes a guide beam irradiation unit 110, a reflection unit 120, a laser irradiation unit 130, an image acquisition unit 140, and an image mapping unit 150.

The guide beam irradiation unit 110 irradiates a guide beam to an eyeball 10 of a patient 20. The guide beam is irradiated toward the retina of the eyeball 10, and is irradiated to a preset position of the retina.

The guide beam irradiation unit 110 includes a guide beam irradiation member 111 and a guide beam body 112.

The guide beam irradiation member 111 irradiates the guide beam toward the eyeball 10. For this purpose, the guide beam irradiation member 111 may include a lens.

The guide beam body 112 is coupled to the guide beam irradiation member 111, and generates a guide beam and delivers the guide beam to the guide beam irradiation member 111.

Using the guide beam irradiation member 111 and the guide beam body 112 described above, an operator positions the patient 20 so that the eyeball 10 of the patient 20 faces the guide beam irradiation member 111, and then irradiates the guide beam to the eyeball 10 by operating the guide beam irradiation member 111.

The reflection unit 120 is positioned adjacent to the guide beam irradiation unit. Specifically, the reflection unit 120 is positioned on the same straight line as the guide beam irradiation member 111. In addition, a reflective mirror is formed inside the reflection unit 120 to refract the reflection beam reflected from the eyeball 10 and transmit the reflection beam to the image acquisition unit 140.

The laser irradiation unit 130 irradiates a laser to the eyeball 10. Specifically, the laser irradiation unit 130 irradiates the laser by changing an irradiation angle and an irradiation position. The reason for changing the irradiation angle and irradiation position of the laser as described above is to acquire retinal images for different positions of the retina.

Accordingly, at least one retinal image is a retinal image at a different angle and position.

Here, at least one retinal image includes an irradiation position of the laser.

The laser irradiation unit 130 includes a laser irradiation member 131, a laser control member 132, and a connection member 133.

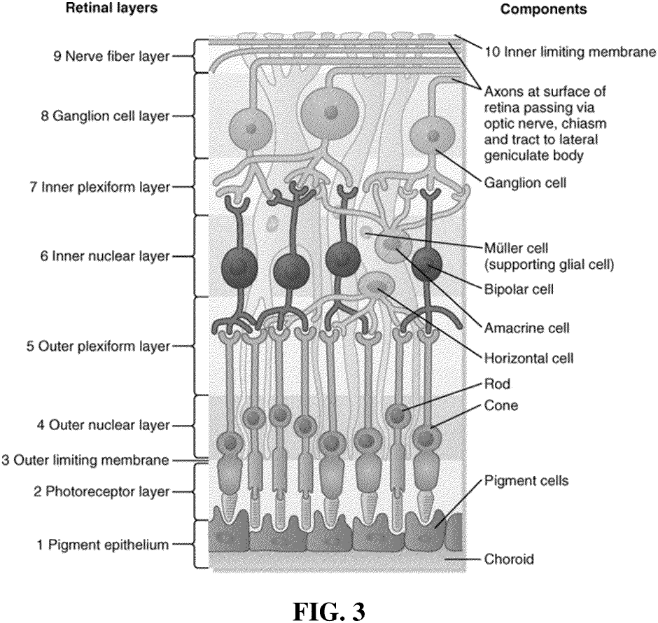

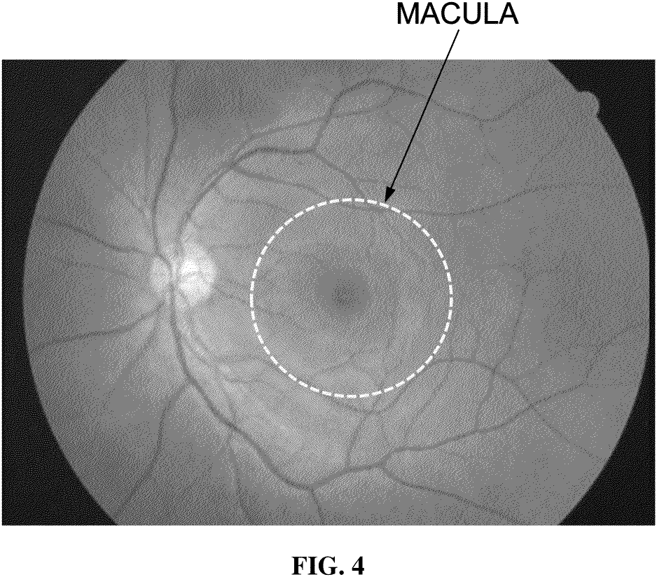

FIG. 3 is a structural view showing 10 layers of a retina for describing a laser feedback device for irradiating an eyeball according to an embodiment of the present invention. FIG. 4 is a view showing an eyeball and macula for describing a laser feedback device for irradiating an eyeball according to an embodiment of the present invention.

The laser irradiation member 131 is positioned to be parallel to the reflection unit 120 and irradiates the laser toward the eyeball 10. The laser irradiation member 131 irradiates the laser to the eyeball 10 after the guide beam is irradiated from the guide beam irradiation member 111.

In this case, the laser irradiates only the pigment epithelium including the pigment cell and choroid of the retina as shown in FIG. 3.

The laser in the present invention has a lower energy level than a laser having a higher energy level used in the prior art. Since the laser used in the prior art has a high energy level, when it is irradiated to the retina, a laser burn is formed on the entire retinal layer shown in FIG. 3 to damage the macula of the retina shown in FIG. 4, and thus, the patient is more likely to lose vision.

However, since the laser irradiated from the laser irradiation unit 130 of the present invention has a low energy level, only the pigment epithelium is locally irradiated, and thus, a laser burn is not formed in the entire retinal layer, so that the patient is protected from vision loss.

Whether the laser irradiation member 131 is irradiated or not is determined by an on/off switch of the laser control member 132.

The guide beam irradiation member 111, the reflection unit 120 and the laser beam irradiation member 131 are positioned to be parallel to each other.

The laser control member 132 controls the operation of the laser. Specifically, the laser control member 132 may be, for example, a foot switch capable of controlling an on/off operation of the laser irradiation member 131.

Specifically, the laser control member 132 controls the operation of the laser irradiated from the laser irradiation member 131 as an operation signal is transmitted to the laser irradiation member 131 through a connection member 133.

The connection member 133 electrically connects the laser irradiation member 131 and the laser control member 132. Specifically, the connection member 133 may be a covered wire, and transmits a corresponding operation signal to the laser irradiation member 131 according to the on-off operation of the laser control member 132.

The image acquisition unit 140 receives the reflection beam reflected from the eyeball 10 and refracted through the reflection unit 120 to acquire at least one retinal image of the retina of the eyeball 10.

Specifically, when the laser is irradiated while the guide beam is irradiated to the eyeball 10, the image acquisition unit 140 acquires at least one retinal image.

The image acquisition unit 140 includes a convex lens 141, a filter member 142, a concave lens 143, and an image processing member 144.

The convex lens 141 is positioned under the reflection unit 120 to receive and focus the reflection beam refracted by the reflection unit 120, and since the convex lens 141 corresponds to the prior art, a detailed description thereof will be omitted.

The filter member 142 is positioned under the convex lens 141 to filter the reflection beam focused by the convex lens 141, and since this filter member 142 corresponds to the prior art, a detailed description thereof will be omitted.

The concave lens 143 is positioned under the filter member 142 to disperse the reflection beam filtered by the filter member 142.

The image processing member 144 is positioned under the concave lens 143 and acquires at least one retinal image based on the reflection beam dispersed from the concave lens 143. As an example, the image processing member 144 may be CMOS, but is not limited thereto, and any image processing member may be used.

The convex lens 141, the filter member 142, the concave lens 143, and the image processing member 144 are disposed perpendicular to the guide beam irradiation unit 110, the reflection unit 120, and the laser irradiation unit 130..

The image mapping unit 150 maps at least one retinal image to construct a macula image that is an entire image of the macula of the eyeball 10. Here, the macula image includes the irradiation position of the laser, and the image mapping unit 150 records the irradiation position of the laser.

The image mapping unit 150 constructs the macula image by mapping at least one retinal image based on at least one of the optic nerve, blood vessel, and discolored region of the eyeball 10 captured as the at least one retinal image.

Specifically, when the image mapping unit 150 irradiates the laser from the laser irradiation unit 130 to the eyeball at different irradiation angles and irradiation positions, the image mapping unit acquires at least one retinal image each time and then maps the retinal image to construct the macula image.

Accordingly, the operator can determine the lesion position in the eyeball 10 of the patient 20 who has been treated once by examining the mapped macula image, so that the operator can perform the treatment in a place other than the treated lesion.

Hereinafter, a laser feedback method using the laser feedback device for irradiating an eyeball according to an embodiment of the present invention will be described with reference to FIGS. 1 to 4.

A laser feedback method which uses the laser feedback device for irradiating an eyeball according to an embodiment of the present invention includes the steps of (a) positioning the eyeball 10 of a patient so that the eyeball 10 is positioned on the same straight line as the guide beam irradiation unit 110, (b) irradiating the guide beam toward the eyeball 10 by the guide beam irradiation unit 110, (c) irradiating the laser toward the eyeball 10 by the laser beam irradiation unit 130, (d) refracting the reflection beam reflected from the eyeball 10 and reflecting the reflection beam to the image acquisition unit 140 by the reflection unit, (e) receiving the reflection beam reflected from the eyeball 10 and refracted through the reflection unit and acquiring at least one retinal image for the retina of the eyeball 10 by the image acquisition unit 140, and (f) mapping the at least one retinal image to construct a macula image, which is the entire image of the macula of the eyeball 10 by the image mapping unit 150.

In addition, the present invention may further include the step of acquiring, by the image acquisition unit 140, an image obtained by capturing the position of the guide beam before step (c). In this step, since the amount of light of the treatment laser is very intense to take an image and it is difficult to actually acquire a retinal image, it is performed to utilize the irradiation position of the guide beam with a relatively weak light intensity.

The step (e) includes the steps of (e1) focusing the reflection beam by the convex lens 141 of the image acquisition unit 140, (e2) filtering the reflection beam focused by the filter member 142 of the image acquisition unit 140, (e3) dispersing the reflection beam filtered by the concave lens 143 of the image acquisition unit 140, and (e4) acquiring the at least one retinal image for the retina of the eyeball 10 based on the reflection beam dispersed by the image processing member 144 of the image acquisition unit 140.

Here, the at least one retinal image may include an irradiation position of the laser.

Next, the step (f) includes the steps of (f1) receiving the at least one retinal image by the image mapping unit 150, (f2) mapping the at least one retinal image based on at least one of the optic nerve, blood vessel, and discolored region of the eyeball 10 captured as the at least one retinal image by the image mapping unit 150, and constructing the macula image based on the at least one retinal image mapped by the image mapping unit 150. The macula image includes the irradiation position of the laser, and the image mapping unit 150 records the irradiation position of the laser.

The foregoing description of the present invention is for illustration, and those of ordinary skill in the art to which the present invention pertains can understand that it can be easily modified into other specific forms without changing the technical spirit or essential features of the present invention. Therefore, it should be understood that the embodiments described above are illustrative in all respects and not restrictive. For example, each component described as a single type may be implemented in a distributed manner, and likewise components described as distributed may also be implemented in a combined form.

The scope of the present invention is indicated by the following claims, and all changes or modifications derived from the meaning and scope of the claims and their equivalents should be construed as being included in the scope of the present invention.

Description of Reference Numerals

| | |
|---|---|
| 10 | eyeball |
| 20 | patient |
| 100 | laser feedback device for irradiating an eyeball |
| 110 | guide beam irradiation unit |
| 111 | guide beam irradiation member |
| 112 | guide beam member |
| 120 | reflection unit |
| 130 | laser irradiation unit |
| 131 | laser irradiation member |
| 132 | laser control member |
| 133 | connection member |
| 140 | image acquisition unit |
| 141 | convex lens |
| 142 | filter member |
| 143 | concave lens |
| 144 | image processing member |
| 150 | imaging mapping unit |

1. A laser feedback device for irradiating an eyeball, comprising:
a guide beam irradiation unit configured to irradiate a guide beam toward an eyeball of a patient;
a reflection unit positioned adjacent to the guide beam irradiation unit;
a laser irradiation unit configured to irradiate a laser toward the eyeball;
an image acquisition unit configured to receive a reflection beam reflected from the eyeball and refracted through the reflection unit and acquire at least one retinal image of a retina of the eyeball; and
an image mapping unit configured to map the at least one retinal image to construct a macular image which is a full image of a macula of the eyeball.

2. The laser feedback device according to claim 1, wherein the laser irradiation unit irradiates the laser by changing an irradiation angle and an irradiation position, and the at least one retinal image is a retinal image at a different angle and position.

3. The laser feedback device according to claim 2, wherein the image mapping unit constructs the macula image by mapping the at least one retinal image based on at least one of an optic nerve, a blood vessel, and a discolored region of the eyeball captured as the at least one retinal image.

4. The laser feedback device according to claim 3, wherein the at least one retinal image and the macula image include the irradiation position of the laser, and the image mapping unit records the irradiation position of the laser.

5. The laser feedback device according to claim 1, wherein the laser irradiates only a pigment epithelium including a pigment cell and a choroid of the retina.

6. The laser feedback device according to claim 1, wherein when the laser is irradiated while the guide beam is irradiated to the eyeball, the image acquisition unit acquires the at least one retinal image.

7. The laser feedback device according to claim 1, wherein the laser irradiation unit includes:
a laser irradiation member that is positioned parallel to the reflection unit and irradiates the laser toward the eyeball;
a laser control member that controls an operation of the laser; and
a connection member that electrically connects the laser irradiation member and the laser control member,
wherein the laser control member controls the operation of the laser irradiated from the laser irradiation member as an operation signal is transmitted to the laser irradiation member through the connection member.

8. The laser feedback device according to claim 7, wherein the guide beam irradiation unit includes:
a guide beam irradiation member that irradiates the guide beam toward the eyeball; and
a guide beam body that is coupled to the guide beam irradiation member, generates the guide beam and delivers the guide beam to the guide beam irradiation member,
wherein the guide beam irradiation member, the reflection unit, and the laser beam irradiation member are positioned to be parallel to each other.

9. The laser feedback device according to claim 1, wherein the image acquisition unit includes:
a convex lens that is positioned under the reflection unit to receive and focus the reflection beam refracted by the reflection unit;
a filter member that is positioned under the convex lens to filter the reflection beam focused by the convex lens;
a concave lens that is positioned under the filter member to disperse the reflection beam filtered by the filter member; and
an image processing member that is positioned under the concave lens to acquire the at least one retinal image based on the reflection beam dispersed from the concave lens,
wherein the convex lens, the filter member, the concave lens, and the image processing member are disposed to be perpendicular to the guide beam irradiation unit, the reflection unit, and the laser irradiation unit.

10. A laser feedback method using a laser feedback device for irradiating an eyeball, the laser feedback method comprising:
(a) positioning an eyeball of a patient so that the eyeball is positioned on a same straight line as a guide beam irradiation unit;
(b) irradiating a guide beam toward the eyeball by the guide beam irradiation unit;
(c) irradiating a laser toward the eyeball by a laser beam irradiation unit;
(d) refracting a reflection beam reflected from the eyeball and reflecting the reflection beam to an image acquisition unit by a reflection unit;
(e) receiving the reflection beam reflected from the eyeball and refracted through the reflection unit and acquiring at least one retinal image for a retina of the eyeball by an image acquisition unit; and
(f) mapping the at least one retinal image to construct a macula image, which is an entire image of a macula of the eyeball by an image mapping unit.

11. The laser feedback method according to claim 10, wherein the step (e) includes:
(e1) focusing the reflection beam by a convex lens of the image acquisition unit;
(e2) filtering the reflection beam focused by a filter member of the image acquisition unit;
(e3) dispersing the reflection beam filtered by a concave lens of the image acquisition unit; and
(e4) acquiring the at least one retinal image for the retina of the eyeball based on the reflection beam dispersed by an image processing member of the image acquisition unit,
wherein the at least one retinal image includes an irradiation position of the laser.

12. The laser feedback method according to claim 10, wherein the step (f) includes:
(f1) receiving the at least one retinal image by the image mapping unit;
(f2) mapping the at least one retinal image based on at least one of an optic nerve, a blood vessel, and a discolored region of the eyeball captured as the at least one retinal image by the image mapping unit; and
constructing the macula image based on the at least one retinal image mapped by the image mapping unit,
wherein the macula image includes an irradiation position of the laser, and the image mapping unit records the irradiation position of the laser.

13. The laser feedback method according to claim 10, further comprising acquiring, by the image acquisition unit, an image obtained by capturing a position of the guide beam before the step (c).
